(19) United States
(12) Patent Application Publication
(10) Pub. No.: US 2003/0204860 A1
(43) Pub. Date: Oct. 30, 2003

(54) Method for selecting cell lines to be used for nuclear transfer in mammalian species

(76) Inventors: David Melican, Fiskdale, MA (US); Robin E. Butler, Spencer, MA (US); William G. Gavin, Dudley, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/396,890

(22) Filed: Mar. 25, 2003

Related U.S. Application Data
(60) Provisional application No. 60/369,009, filed on Apr. 1, 2002.

(51) Int. Cl. A01K 67/027

(52) U.S. Cl. 800/14; 800/15; 800/16; 800/17

(57) ABSTRACT

The present invention provides data to demonstrate that the fusion performance of a cell-line in procedures involving fusion and cleavage indices either alone or in combination are a means for selecting a cell lines that will be successful in a nuclear transfer or microinjection program. This technique and method of selecting a cell line offers an additional alternative and improvement in the creation of activated and fused nuclear transfer-capable embryos for the production of live offspring in various mammalian non-human species including goats, pigs, rodents, primates, rabbits and cattle.

### FIELD OF THE INVENTION

The present invention relates to improved methods for the selection of a superior cell line or lines to be used in nuclear transfer or nuclear microinjection procedures in non-human mammals. More specifically, the current invention provides a method to improve the results in such transgenic programs by providing criteria that enable the pre-selection of a superior cell line.

### BACKGROUND OF THE INVENTION

The present invention relates generally to the field of somatic cell nuclear transfer (SCNT) and to the creation of desirable transgenic animals. More particularly, it concerns methods for selecting, generating, and propagating superior somatic cell-derived cell lines, transforming these cell lines, and using these transformed cells and cell lines to generate transgenic non-human mammalian animal species. Typically these transgenic animals will be used for the production of molecules of interest, including biopharmaceuticals, antibodies and recombinant proteins.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, are time-consuming, costly and unreliable. Moreover, these processes are completely incapable of allowing a specific animal line from producing gene products, such as desirable protein therapeutics that are otherwise entirely absent from the genetic complement of the species in question (i.e., human or humanized antibodies in bovine milk).

The development of technology capable of generating transgenic animals provides a means for exceptional precision in the production of animals that are engineered to carry specific traits or are designed to express certain proteins or other molecular compounds of therapeutic or commercial value. That is, transgenic animals are animals that carry a gene that has been deliberately introduced into existing somatic cells and/or germline cells at an early stage of development. As the animals develop and grow the protein product or specific developmental change engineered into the animal becomes apparent.

At present the techniques available for the generation of transgenic domestic animals are inefficient and time-consuming typically producing a very low percentage of viable embryos, often due to poor cell line selection techniques or poor viability of the cells that are selected.

During the development of a transgene, DNA sequences are typically inserted at random in the genetic complement of the target cell nuclei, which can cause a variety of problems. The first of these problems is insertional inactivation, which is inactivation of an essential gene due to disruption of the coding or regulatory sequences by the incoming DNA. Another problem is that the transgene may either be not incorporated at all, or incorporated but not expressed. A further problem is the possibility of inaccurate regulation due to positional effects in the genetic material. This refers to the variability in the level of gene expression and the accuracy of gene regulation between different founder animals produced with the same transgenic constructs. Thus, it is not uncommon to generate a large number of founder animals and often confirm that less than 5% express the transgene in a manner that warrants the maintenance of that transgenic line.

Additionally, the efficiency of generating transgenic domestic animals is low, with efficiencies of 1 in 100 offspring generated being transgenic not uncommon (Wall, 1997). As a result the cost associated with generation of transgenic animals can be as much as 250-500 thousand dollars per expressing animal (Wall, 1997).

Prior art methods of nuclear transfer and microinjection have typically used embryonic and somatic cells and cell lines selected without regard to any objective factors tying cell quality relative to the procedures necessary for transgenic animal production. This type of work and cell sourcing is typified by Campbell et al (Nature, 1996) and Stice et al (Biol. Reprod., 1996). In both of those studies, cell lines were derived from embryos of less than 10 days of gestation. In both studies, the cells selected were maintained on a feeder layer to prevent overt differentiation of the donor cell to be used in the cloning procedure, but no other selection method, technique or procedure was used. The present invention uses differentiated cells selected for their suitability for nuclear transfer and microinjection procedures as a source of karyoplasts based on their performance in at least one objective test of suitability. The current invention also contemplates the use of embryonic cell types could also be screened using the methods of the current invention along with cloned embryos starting with differentiated donor nuclei.

Thus although transgenic animals have been produced by various methods in several different species, methods to readily and reproducibly produce transgenic animals capable of expressing the desired protein in high quantity or demonstrating the genetic change caused by the insertion of the transgene(s) at reasonable costs are still lacking.

Accordingly, a need exists for improved methods of selecting cell lines as the source for karyoplasts in nuclear transfer procedures that will allow an increase in production efficiencies in the development of transgenic animals. The current invention then enhances the ability to select a cell line that is optimal for nuclear transfer or microinjection procedures. Currently, there are quite a large degree of successes and failures that can be attributed to inferior cell lines being used as the source of karyoplasts in nuclear transfer procedures, the current invention will improve these efficiencies.

### SUMMARY OF THE INVENTION

Briefly stated, the current invention provides for an improved method for cloning a non-human mammal through a nuclear transfer process comprising: obtaining a desired differentiated mammalian cell line to be used as a source of donor nuclei for nuclear transfer procedures; obtaining at least one oocyte from a mammal of the same species as the cells which are the source of donor nuclei; enucleating the at least one oocyte; transferring the desired differentiated cell or cell nucleus into the enucleated oocyte; simultaneously fusing and activating the cell couplet to form a first transgenic embryo; activating a cell-couplet that does not fuse to create a first transgenic embryo; culturing the activated first transgenic embryo until greater than the 2-cell developmental stage; and transferring the first transgenic embryo into a suitable host mammal such that the embryo develops into a fetus wherein the desired differentiated mammalian cell line to be used as a karyoplast is selected according to the objective parameters of cleavage and/or fusion patterns. Typically, the above method is completed through the use of a donor cell nuclei in which a desired gene has been inserted, removed or modified prior to insertion of said differentiated mammalian cell or cell nucleus into said enucleated oocyte. Also of note is the fact that the oocytes used are preferably matured in vitro prior to enucleation.

Moreover, the method of the current invention also provides for optimizing the generation of transgenic animals through the use of caprine oocytes, arrested at the Metaphase-II stage, that were enucleated and fused with donor somatic cells and simultaneously activated. Analysis of the milk of one of the transgenic cloned animals showed high-level production of human of the desired target transgenic protein product.

It is also important to point out that the present invention can also be used to increase the availability of CICM cells, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimericism can be used to avoid immunological rejection among animals of the same species as well as between species.

### BRIEF DESCRIPTION OF THE DRAWINGS

FIG. 1 Shows A Generalized Diagram of the Process of Creating Cloned Animals through Nuclear Transfer.

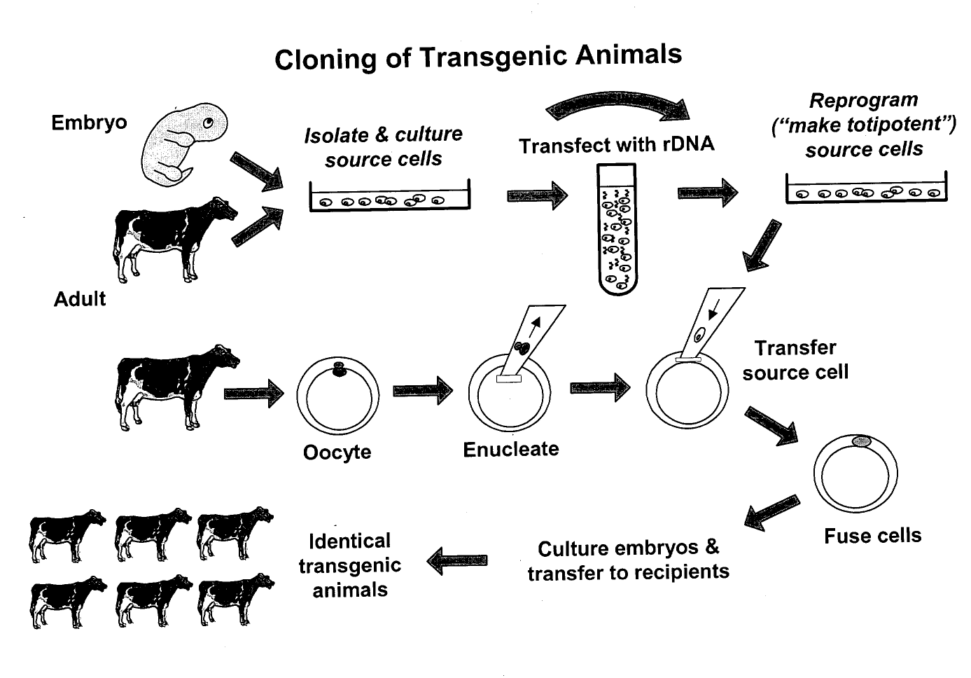

### DESCRIPTION OF THE PREFERRED EMBODIMENT

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:

Bovine—Of or relating to various species of cows.

Caprine—Of or relating to various species of goats.

Cell Couplet—An enucleated oocyte and a somatic or fetal karyoplast prior to fusion and/or activation.

Cytocholasin-B—A metabolic product of certain fungi that selectively and reversibly blocks cytokinesis while not effecting karyokinesis.

Cytoplast—The cytoplasmic substance of eukaryotic cells.

Fusion Slide—A glass slide for parallel electrodes that are placed a fixed distance apart. Cell couplets are placed between the electrodes to receive an electrical current for fusion and activation.

Karyoplast—A cell nucleus, obtained from the cell by enucleation, surrounded by a narrow rim of cytoplasm and a plasma membrane.

Nuclear Transfer—or “nuclear transplantation” refers to a method of cloning wherein the nucleus from a donor cell is transplanted into an enucleated oocyte.

Parthenogenic—The development of an embryo from an oocyte without the penetrance of sperm

Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.

Somatic Cell—Any cell of the body of an organism except the germ cells.

Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

Transgenic Organism—An organism into which genetic material from another organism has been experimentally transferred, so that the host acquires the genetic traits of the transferred genes in its chromosomal composition.

According to the present invention, multiplication of superior genotypes of mammals with enhanced efficiencies, including caprines and bovines, is provided. This will allow the multiplication of adult animals with proven genetic superiority or other desirable traits, superiority here including successful performance in objective tests of cell quality and suitability for the production of transgenic animals. Progress will be enhanced, for example, in the success rates of generation of many important mammalian species including goats, rodents, cows and rabbits. By the present invention, there are potentially billions of fetal or adult cells that can be harvested and used in the cloning procedure and that will then be tested according to objective parameters to indicate suitability for the procedures, methods and techniques necessary for the production of transgenic animals. This will potentially result in many identical offspring in a short period, decreasing overall costs involved and improving efficiencies.

In addition, the present invention relates to cloning procedures in which cell nuclei derived from somatic or differentiated fetal or adult mammalian cell lines are utilized. These cell lines include the use of serum starved differentiated fetal or adult caprine or bovine (as the case may be) cell populations and cell lines later re-introduced to serum as mentioned infra, these cells are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce chimeric embryos, fetuses and/or offspring.

Wilmut et al. (1997), although earlier reported by Campbell et al. (1996), reported fusion rate and embryo development for their successful cloning work but did not document that either or both of these parameters were significant for one cell line being statistically significantly superior to another. Numerous other studies have continued to report only fusion rate (Kasinathan et al., 2001; Lai et al., 2001; Keefer et al., 2002; Reggio et al., 2001; and Fitchev et al., 1999), fusion and cleavage (Kato et al., 2000; Zakhartchenko et al., 1996; Zakhartchenko et al., 2001; Verma et al., 2000; Liu et al., 2001; Park et al., 2001; and Booth et al., 2001) or cleavage without fusion (Kuholzer et al., 2001; Zou et al., 2002; and Kou et al., 2000). These reports again did not indicate or address that a given cell line was superior for use as a source of karyoplasts in nuclear transfer procedures based on statistically significant higher rates of fusion and/or cleavage.

The current invention also provides for the enhancement of efficiencies in somatic cell nuclear transfer through the simultaneous fusion and activation with no delay involved between the two events. The purpose of this current study was to investigate the link between fusion and/or cleavage as an indicator of cell line potential for use in producing viable offspring in a nuclear transfer program.

Fusion of a donor karyoplast to an enucleated cytoplast, and subsequent activation of the resulting couplet are important steps required to successfully generate live offspring by somatic cell nuclear transfer. Electrical fusion of a donor karyoplast to a cytoplast is the most common method used. More importantly however, several methods of activation, and the timing of the activation steps, used in nuclear transfer methodologies to initiate the process of embryo development in numerous livestock species have been published. In mammals, while there are species differences, the initial signaling events and subsequent Ca⁺² oscillations induced by sperm at fertilization are the normal processes that result in oocyte activation and embryonic development (Fissore et al., 1992 and Alberio et al., 2001). Both chemical and electrical methods of Ca⁺² mobilization are currently utilized to activate couplets generated by somatic cell nuclear transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al, Biol. Reprod. 2001; Lai et al., 2001; Yong et al., 1998; and Kasinathan et al., Nature Biotech 2001).

Prior art techniques rely on the use of randomly sourced blastomeres of early embryos for nuclear transfer procedure. This approach is limited by the small numbers of available embryonic blastomeres and by the inability to introduce foreign genetic material into such cells. In contrast, the discoveries that differentiated embryonic, fetal, or adult somatic cells can function as karyoplast donors for nuclear transfer have provided a wide range of possibilities for germline modification. According to the current invention, the use of recombinant somatic cell lines for nuclear transfer, and improving this procedures efficiency by selecting superior cell lines that can be more successfully used in nuclear transfer methods including use of “reconstructed” embryos, not only enhances the efficiency of traditional transfection methods but also increases the efficiency of transgenic animal production substantially while overcoming the problem of founder mosaicism.

We have previously shown that simultaneous electrical fusion and activation can successfully produce live offspring in the caprine species, and other animals. In a recent set of experiments, we investigated the use of additional electrical activation events, following initial successful simultaneous electrical fusion and activation, to more closely mimic sperm-induced Ca⁺² oscillations and generate both embryos and live offspring by somatic cell nuclear transfer. Finally, we determined the ability of re-fusing donor karyoplasts to enucleated cytoplasts, which did not successfully fuse at the initial simultaneous electrical fusion and activation event, to generate both goat embryos and live offspring by somatic cell nuclear transfer.

The efficiency of electrical fusion of a karyoplast to an enucleated cytoplast varies based on species and the cell type used. However, in our experience with the goat, and as reported by others (Baguisi et al., 1999; and Stice et al., 1992), there is a sub-population of couplets that do not successfully fuse during the initial fusion attempt. In these experiments, we determined the ability of an additional re-fusion attempt following an unsuccessful initial simultaneous electrical fusion and activation event to generate both goat embryos and live offspring by somatic cell nuclear transfer. In experiments, the data demonstrates that re-fusion was both capable and more efficient, compared to simultaneous electrical fusion and activation alone (Baguisi et al., 1999), or a single additional electrical activation event following the initial successful simultaneous electrical fusion and activation, in the ability to produce live offspring. In subsequent experiments, we confirmed our observations that re-fusion of non-fused couplets were able to generate nuclear transfer embryos capable of establishing pregnancies at day 55 of gestation.

Thus, through the methodology and system employed in the current invention transgenic animals, goats, transgenic animals have been generated by somatic cell nuclear transfer whose efficiencies were enhanced through the use of objective cell selection criteria.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Wilmut et al., and Campbell et al., reported using a single electrical pulse for fusion of the reconstructed embryo followed by a delay for a number of hours prior to activation of the embryo chemically. Other reports have demonstrated the different electrical and chemical stimuli that could be used for activation in various species (Koo et al., 2000; and Fissore A., et al,). The current invention provides for the use of somatic cell nuclear transfer by simultaneous fusion and activation with no delay involved between the two events, with the use of subsequent additional electrical pulses to an activated and fused embryo. However, the cell selection techniques provided herein will improve a broad range of nuclear transfer techniques, including the more traditional methods provided by Wilmut et al., and Campbell et al., by improving the “starting material” or cells used in those process. Likewise the techniques utilized herein with regard to caprine cells and cell lines are also useful in a variety of other mammalian cell lines. The methods of the current invention rely on characteristics of the cells being investigated, namely cleavage and/or fusion as objective criteria, regardless of the species. Thus, the current invention provides nuclear transfer techniques that provide improved efficiencies and make the process of producing transgenic animals or cell lines more reliable and efficient.

### MATERIALS AND METHODS

Estrus synchronization and superovulation of donor does used as oocyte donors, and micro-manipulation was performed as described in Gavin W.G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Enucleation and reconstruction was performed with, but may also be performed without, staining the oocytes with Hoechst 3342 at approximately 0.1-5.0 ug/ml and ultraviolet illumination of the genetic material/metaphase plate. Following enucleation and reconstruction, the karyoplast/cytoplast couplets were incubated in equilibrated Synthetic Oviductal Fluid medium supplemented with fetal bovine serum (1% to 15%) plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

Fusion was performed using a fusion slide constructed of two electrodes. The fusion slide was placed inside a fusion dish, and the dish was flooded with a sufficient amount of fusion buffer to cover the electrodes of the fusion slide. Cell couplets were removed from the culture incubator and washed through fusion buffer. Using a stereomicroscope, cell couplets were placed equidistant between the electrodes, with the karyoplast/cytoplast junction parallel to the electrodes. In these experiments an initial single simultaneous fusion and activation electrical pulse of approximately 2.0 to 3.0 kV/cm for 20 (can be 20-60) μsec was applied to the cell couplets using a BTX ECM 2001 Electrocell Manipulator. The fusion treated cell couplets were transferred to a drop of fresh fusion buffer. Fusion treated couplets were washed through equilibrated SOF/FBS, then transferred to equilibrated SOF/FBS with (1 to 10 μg/ml) or without cytochalasin-B. The cell couplets were incubated at 37-39° C. in a humidified gas chamber containing approximately 5% CO₂ in air.

Starting at approximately 30 minutes post-fusion, karyoplast/cytoplast fusion was determined. Fused couplets received an additional single electrical pulse (double pulse) of approximately 2.0 kV/cm for 20 (20-60) μsec starting at 1 hour (15 min-1 hour) following the initial fusion and activation treatment to facilitate additional activation. Alternatively, another group of fused cell couplets received three additional single electrical pulses (quad pulse) of approximately 2.0 kV/cm for 20 μsec, at fifteen-minute intervals, starting at 1 hour (15 min to 1 hour) following the initial fusion and activation treatment to facilitate additional activation. Non-fused cell couplets were re-fused with a single electrical pulse of approximately 2.6 to 3.2 kV/cm for 20 (20-60) μsec starting at 1 hours following the initial fusion and activation treatment to facilitate fusion. All fused and fusion treated cell couplets were returned to SOF/FBS with (1 to 10 μg/ml) or without cytochalasin-B. The cell couplets were incubated at least 30 minutes at 37-39° C. in a humidified gas chamber containing approximately 5% CO₂ in air.

Starting at 30 minutes following re-fusion, the success of karyoplast/cytoplast re-fusion was determined. Fusion treated cell couplets were washed with equilibrated SOF/FBS, then transferred to equilibrated SOF/FBS with (1 to 10 μg/ml) or without cycloheximide. The cell couplets were incubated at 37-39° C. in a humidified gas chamber containing approximately 5% CO₂ in air for up to 4 hours.

Following cycloheximide treatment, cell couplets were washed extensively with equilibrated SOF medium supplemented with bovine serum albumin (0.1% to 1.0%) plus 100 U/ml penicillin and 100 μg/ml streptomycin (SOF/BSA). Cell couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

The data presented in Table 1 are from the production nuclear transfer work for the production of founder transgenic animals developed in the period from September 2001 through early February 2002. This table details the lab production effort and specifically the embryo collection, enucleation, fusion, cleavage and transfer data.

TABLE 1
Nuclear Transfer Data 2001/2002 Season

| | 2001/2002 Season |
|---|---|
| | (Aug. 27, 2001-Feb. 8, 2002) |
| Total Ovulations | 7151 |
| # Donors | 495 |
| Ovulations/Donor | 14.4 |
| # Ova Retrieved | 4201 (59% of ovulations) |
| # Ova/Donor | 8.5 |
| # Ova ovulated & | 4452 |
| aspirated | |
| # enucleated | 4215 (95% oocytes recovered) |
| # reconstructed | 3947 (94% oocytes enucleated) |
| # couplets fusion | 3633 (92% oocytes reconstructed) |
| attempted | |
| # couplets fused | 2904 (80% fusion attempted) |
| # cleaved | 1145 (39% couplets fused) |
| | (58% @ 48 hrs) |
| # nuclear transfer | 2120 |
| embryos transferred | |
| # Recipients | 345 |
| # Embryos/Recipient | 6.1 (range 1-15) |
| # Pregnancies | 24 (40)/305 (7.9%) through week 19 |
| # Offspring | Pending |

More relevant information for the current invention is found below in Table 2 where the data has been presented based on fusion and cleavage rate as separated by pregnant vs non-pregnant animals indicating that where the rates of fusion and/or cleavage are higher in a given cell population or cell line that cell line has greater overall success in predicting a developing pregnancy and the birth of a transgenic animal.

TABLE 2
Summary of GTC Nuclear Transfer Pregnancies by Fusion and Cleavage

| | NT recipients | |
|---|---|---|
| | US positive | NT recipients |
| | (day 50) | US negative |
| # Recipients | 26 | 139 |
| # Experiments | 17 | 35 |
| # Cell lines | 13 | 15 |
| # Fusion attempted | 826 | 1424 |
| # Fused (%) | 686^{a} (83) | 1093^{b} (77) |
| Fusion range (%) | (57-100) | (32-100) |
| # Cleaved @ 48 hrs/ | 239/339 (71)^{a} | 376/721 (52)^{b} |
| # Fused (%) | | |
| (range %) | (57-92) | (22-93) |

^{a,b}Values within rows with different superscripts differ significantly (P < 0.001).

The ability to pre-select a superior cell line to be used in a nuclear transfer program has remarkable implications. A significant amount of nuclear transfer work occurs with limited success as seen by the publications referenced in this document. In many of these publications a fair amount of work is done with very poor results or a complete lack of offspring born for individual cell (karyoplast) lines.

Paramount to the success of any nuclear transfer program is having adequate fusion of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both fusion and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

According to the current invention the characteristics of a certain cell line or cell population relative to fusion, fusion and cleavage, or cleavage alone in their respective publications, are critical and statistically significant when evaluating a cell line for use in a nuclear transfer program. Going further, elements of the current invention demonstrate that the nuclear index (number of blastomeres from a reconstructed nuclear transfer embryo that have a nucleus) of an embryo is also a relevant indicator of cell line performance.

Essentially, the current invention provides that through the use of fusion and cleavage indices either alone or in combination are a means for selecting superior cell lines useful in enhancing the successful initiation and conclusion of a nuclear transfer program

Goats.

The herds of pure- and mixed-breed scrapie-free Alpine, Saanen and Toggenburg dairy goats used as cell and cell line donors for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I. U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) for at least 6 hours prior to fusion to the enucleated oocytes.

Oocyte Collection.

Oocyte donor does were synchronized and superovulated as previously described (Gavin W.G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 □m glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Starting at between 10 to 90 minutes post-fusion, most preferably at 30 minutes post-fusion, the presence of an actual karyoplast/cytoplast fusion is determined. For the purposes of the current invention fused couplets may receive an additional activation treatment (double pulse). This additional pulse can vary in terms of voltage strength from 0.1 to 5.0 kV/cm for a time range from 10 to 80 μsec. Preferably however, the fused couplets would receive an additional single electrical pulse (double pulse) of 0.4 or 2.0 kV/cm for 20 μsec. The delivery of the additional pulse could be initiated at least 15 minutes hour after the first pulse, most preferably however, this additional pulse would start at 30 minutes to 2 hours following the initial fusion and activation treatment to facilitate additional activation. In the other experiments, non-fused couplets were re-fused with a single electrical pulse. The range of voltage and time for this additional pulse could vary from 1.0 kV/cm to 5.0 kV/cm for at least 10μsec occurring at least 15 minutes following an initial fusion pulse. More preferably however, the additional electrical pulse varied from of 2.2 to 3.2 kV/cm for 20 μsec starting at 30 minutes to 1 hour following the initial fusion and activation treatment to facilitate fusion. All fused and fusion treated couplets were returned to SOF/FBS plus 5 μg/ml cytochalasin-B. The couplets were incubated at least 20 minutes, preferably 30 minutes, at 37-39° C. in a humidified gas chamber containing approximately 5% CO₂ in air.

An additional version of the current method of the invention provides for an additional single electrical pulse (double pulse), preferably of 2.0 kV/cm for the cell couplets, for at least 20 μsec starting at least 15 minutes, preferably 30 minutes to 1 hour, following the initial fusion and activation treatment to facilitate additional activation. The voltage range for this additional activation pulse could be varied from 1.0 to 6.0 kV/cm.

Alternatively, in subsequent efforts the remaining fused couplets received at least three additional single electrical pulses (quad pulse) most preferably at 2.0 kV/cm for 20 μsec, at 15 to 30 minute intervals, starting at least 30 minutes following the initial fusion and activation treatment to facilitate additional activation. However, it should be noted that in this additional protocol the voltage range for this additional activation pulse could be varied from 1.0 to 6.0 kV/cm, the time duration could vary from 10 μsec to 60 μsec, and the initiation could be as short as 15 minutes or as long as 4 hours following initial fusion treatments. In the subsequent experiments, non-fused couplets were re-fused with a single electrical pulse of 2.6 to 3.2 kV/cm for 20 μsec starting at 1 hours following the initial fusion and activation treatment to facilitate fusion. All fused and fusion treated couplets were returned to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air for at least 30 minutes. Mannitol can be used to substitute for Cytocholasin-B.

Starting at 30 minutes following re-fusion, the success of karyoplast/cytoplast re-fusion was determined. Fusion treated couplets were washed with equilibrated SOF/FBS, then transferred to equilibrated SOF/FBS plus 5 μg/ml cycloheximide. The couplets were incubated at 37-39° C. in a humidified gas chamber containing approximately 5% CO₂ in air for up to 4 hours.

Following cycloheximide treatment, couplets were washed extensively with equilibrated SOF medium supplemented with at least 0.1% bovine serum albumin, preferably at least 0.7%, preferably 0.8%, plus 100U/ml penicillin and 100 μg/ml streptomycin (SOF/BSA). Couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

Nuclear Transfer Embryo Culture and Transfer to Recipients.

All nuclear transfer embryos were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2□ (Lutalyse, Upjohn). Parturition occurred within 24 hours after treatment. Kids were removed from the dam immediately after birth, and received heat-treated colostrum within 1 hour after delivery.

Genotyping of Cloned Animals.

Shortly after birth, blood samples and ear skin biopsies were obtained from the cloned female animals (e.g., goats) and the surrogate dams for genomic DNA isolation. Each sample was first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKem®, ME) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with ³²P dCTP using the Prime-It® kit (Stratagene, La Jolla, Calif.). Hybridization was executed at 65° C. overnight. The blot was washed with 0.2 X SSC, 0.1% SDS and exposed to X-OMAT™ AR film for 48 hours.

In the experiments performed during the development of the current invention, following enucleation and reconstruction, the karyoplast/cytoplast couplets were incubated in equilibrated Synthetic Oviductal Fluid medium supplemented with 1% to 15% fetal bovine serum, preferably at 10% FBS, plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

The present invention allows for increased efficiency of transgenic procedures by providing for the use of superior cell in the procedures leading to the generation of transgenic embryos. These transgenic embryos can be implanted in a surrogate animal or can be clonally propagated and stored or utilized. Also by combining enhanced and improved nuclear transfer procedures with the ability to modify and select for these cells in vitro, this procedure is more efficient than previous transgenic embryo techniques. According to the present invention, these transgenic cloned embryos can be used to produce CICM cell lines or other embryonic cell lines. Therefore, the present invention eliminates the need to derive and maintain in vitro an undifferentiated, unselected, random cell line that is conducive to genetic engineering techniques.

Thus, in one aspect, the present invention provides a method for cloning a mammal. In general, a mammal can be produced by a nuclear transfer process comprising the following steps:

(i) obtaining desired differentiated mammalian cells to be used as a source of donor nuclei;

(ii) obtaining oocytes from a mammal of the same species as the cells that are the source of donor nuclei;

(iii) enucleating said oocytes;

(iv) transferring the desired differentiated cell or cell nucleus into the enucleated oocyte;

(v) simultaneously fusing and activating the cell couplet to form a first transgenic embryo;

(vi) continuing the activation a cell-couplet that does not fuse to create a first transgenic embryo by providing a second activating electrical shock to form a second transgenic embryo;

(vii) culturing said activated first and/or second transgenic embryo until greater than the 2-cell developmental stage; and

(viii) transferring said first and/or second transgenic embryo into a host mammal such that the embryo develops into a fetus.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

In another aspect, the present invention provides a method for producing CICM cells. The method comprises:

(i) obtaining desired differentiated mammalian cells to be used as a source of donor nuclei;

(ii) obtaining oocytes from a mammal of the same species as the cells that are the source of donor nuclei;

(iii) enucleating said oocytes;

(iv) transferring the desired differentiated cell or cell nucleus into the enucleated oocyte;

(v) simultaneously fusing and activating the cell couplet to form a first transgenic embryo;

(vi) activating a cell-couplet that does not fuse to create a first transgenic embryo but that is activated after an initial electrical shock by providing at least one additional activation protocol including an additional electrical shock to form a second transgenic embryo;

(vii) culturing said activated first and/or second transgenic embryo until greater than the 2-cell developmental stage; and

(viii) culturing cells obtained from said cultured activated embryo to obtain CICM cells.

Also CICM cells derived from the methods described herein are advantageously used in the area of cell, tissue and organ transplantation, or in the production of fetuses or offspring, including transgenic fetuses or offspring. Differentiated mammalian cells are those cells, which are past the early embryonic stage. Differentiated cells may be derived from ectoderm, mesoderm or endoderm tissues or cell layers.

An alternative method can also be used, one in which the cell couplet can be exposed to multiple electrical shocks to enhance fusion and activation. In general, the mammal will be produced by a nuclear transfer process comprising the following steps:

(i) obtaining desired differentiated mammalian cells to be used as a source of donor nuclei;

(ii) obtaining oocytes from a mammal of the same species as the cells that are the source of donor nuclei;

(iii) enucleating said oocytes;

(iv) transferring the desired differentiated cell or cell nucleus into the enucleated oocyte;

employing at least two electrical shocks to a cell-couplet to initiate fusion and activation of said cell-couplet into an activated and fused embryo.

(vii) culturing said activated and fused embryo until greater than the 2-cell developmental stage; and

(viii) transferring said first and/or second transgenic embryo into a host mammal such that the embryo develops into a fetus;

wherein the second of said at least two electrical shocks is administered at least 15 minutes after an initial electrical shock.

Mammalian cells, including human cells, may be obtained by well-known methods. Mammalian cells useful in the present invention include, by way of example, epithelial cells, neural cells, epidermal cells, keratinocytes, hematopoictic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body and will be screened according to their performance in fusion and/or cleavage studies. This method would then provide for overall increases in transgenic animal generation.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures, and an objective screen or multiple screening techniques as provided for by the current invention. Again the present invention is novel because differentiated cell types are used. The present invention is advantageous because the cells can be easily propagated, genetically modified and selected in vitro.

Suitable mammalian sources for oocytes include goats, sheep, cows, pigs, rabbits, guinea pigs, mice, hamsters, rats, primates, etc. Preferably, the oocytes will be obtained from caprines and ungulates, and most preferably goats. Methods for isolation of oocytes are well known in the art. Essentially, this will comprise isolating oocytes from the ovaries or reproductive tract of a mammal, e.g., a goat. A readily available source of goat oocytes is from hormonal induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields).

The stage of maturation of the oocyte at enucleation and nuclear transfer has been reported to be significant to the success of nuclear transfer methods. (First and Prather 1991). In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially goats, the oocyte activation period generally occurs at the time of sperm contact and penetrance into the oocyte plasma membrane.

After a fixed time maturation period, which ranges from about 10 to 40 hours, and preferably about 16-18 hours, the oocytes will be enucleated. Prior to enucleation the oocytes will preferably be removed and placed in EMCARE media containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in EMCARE media, preferably containing 7.5 micrograms per milliliter cytochalasin B, for immediate enucleation, or may be placed in a suitable medium, for example an embryo culture medium such as CR1aa, plus 10% FBS, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in EMCARE or SOF, and then viewing the oocytes under ultraviolet irradiation for less than 10 seconds. The oocytes that have been successfully enucleated can then be placed in a suitable culture medium.

In the present invention, the recipient oocytes will preferably be enucleated at a time ranging from about 10 hours to about 40 hours after the initiation of in vitro or in vivo maturation, more preferably from about 16 hours to about 24 hours after initiation of in vitro or in vivo maturation, and most preferably about 16-18 hours after initiation of in vitro or in vivo maturation.

A single mammalian cell of the same species as the enucleated oocyte will then be transferred into the perivitelline space of the enucleated oocyte used to produce the activated embryo. The mammalian cell and the enucleated oocyte will be used to produce activated embryos according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,994,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Ponimaskin et al., 2000).

Also, in some cases (e.g. with small donor nuclei) it may be preferable to inject the nucleus directly into the oocyte rather than using electroporation fusion. Such techniques are disclosed in Collas and Barnes, Mol. Reprod. Dev., 38:264-267 (1994), incorporated by reference in its entirety herein.

The activated embryo may be activated by known methods. Such methods include, e.g., culturing the activated embryo at sub-physiological temperature, in essence by applying a cold, or actually cool temperature shock to the activated embryo. This may be most conveniently done by culturing the activated embryo at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may best be effected by simultaneously, although protocols for sequential activation do exist with cell lines selected for their superiority. In terms of activation the following cellular events occur:

(i) increasing levels of divalent cations in the oocyte, and

(ii) reducing phosphorylation of cellular proteins in the oocyte.

The above events can be exogenously stimulated to occur by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation may be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

Accordingly, it is to be understood that the embodiments of the invention herein providing for an increased availability of activated and fused “reconstructed embryos” are merely illustrative of the application of the principles of the invention. It will be evident from the foregoing description that changes in the form, methods of use, and applications of the elements of the disclosed method for the improved selection of cell or cell lines for use in nuclear transfer or microinjeciton procedures are novel and may be modified and/or resorted to without departing from the spirit of the invention, or the scope of the appended claims.

### LITERATURE CITED AND INCORPORATED BY REFERENCE

1. Alberio R, et al., Mammalian Oocyte Activation: Lessons from the Sperm and Implications for Nuclear Transfer, INT J DEV BIOL 2001; 45: 797-809.

2. Alberio R, et al., Remodeling of Donor Nuclei, DNA Synthesis, and Ploidy of Bovine Cumulus Cell Nuclear Transfer Embryos: Effect of Activation Protocol, MOL REPROD DEV 2001; 59: 371-379.

3. Baguisi A, et al., Production of Goats by Somatic Cell Nuclear Transfer, NAT BIOTECH 1999; 17: 456-461.

4. Booth P J, et al., Effect of Two Activation Treatments and Age of Blastomere Karyoplasts on In Vitro Development of Bovine Nuclear Transfer Embryos, MOL REPROD DEV 2001; 60: 377-383.

5. Bondioli K, et al., Cloned Pigs from Cultured Skin Fibroblasts Derived from A H-Transferase Transgenic Boar, MOL REPROD DEV 2001; 60: 189-195.

6. Bondioli K, et al., Production of Identical Bovine Offspring by Nuclear Transfer, THERIOGENOLOGY 1990; 33: 165-174.

7. Campbell, K H S, et al., Sheep Cloned by Nuclear Transfer From a Cultured Cell Line, NATURE 1996; 380: 64-66.

8. Cibelli J B, et al., Cloned Transgenic Calves Produced From Nonquiescent Fetal Fibroblasts. SCIENCE 1998; 280: 1256-1258.

9. Collas P, and Barnes F L., Nuclear Transplantation by Microinjection of Inner Cell Mass and Granulosa Cell Nuclei, MOL REPROD DEV. 1994 July;38(3):264-7.

10. Collas P. Electrically Induced Calcium Elevation, Activation, and Parthenogenic Development of Bovine Oocytes. MOL REPROD 1993; 34: 212-223.

11. Ducibella T., Biochemical and Cellular Insights Into the Temporal Window of Normal Fertilization, THERIO 1998: 49: 53-65.

12. Edmunds, T. et al., Transgenically Produced Human Antithrombin—Structural and Functional Comparison to Human Plasma-Derived Antithrombin, BLOOD 1998; 91:4561-4571.

13. First N L, et al., Genomic Potential in Mammals, DIFFERENTIATION 1991 September;48(1):1-8.

14. Fitchev P, et al., Nuclear Transfer in the Rat: Potential Access to the Germline. TRANSPLANT PROCEED. 1999; 31: 1525-1530.

15. Kasinathan P, et al., Effect of Fibroblast Donor Cell Age and Cell Cycle on Development of Bovine Nuclear Transfer Embryos In Vitro, BIOL REPROD 2001; 64(5): 1487-1493.

16. Kasinathan P, et al., Production of Calves from GI Fibroblasts, NATURE BIOTECH 2001; 19: 1176-1178.

17. Kato Y. et al., Cloning of Calves from Various Somatic Cell Types of Male and Female Adult, Newborn and Fetal Cows, J REPROD FERT 2000; 120: 231-237.

18. Keefer C L, et aL, Production Of Cloned Goats After Nuclear Transfer Using Adult Somatic Cells, BIOL REPROD 2002; 66: 199-203.

19. Koo D B, et al., In Vitro Development of Reconstructed Porcine Oocytes after Somatic Cell Nuclear Transfer. BIOL REPROD 2000; 63: 986-992.

20. Kuhholzer B, et al., Clonal Lines Of Transgenic Fibroblast Cells Derived From The Same Fetus Result In Different Development When Used For Nuclear Transfer In Pigs, BIOL REPROD 2001; 64: 1695-1698.

21. Lai, L, et al., Feasibility of Producing Porcine Nuclear Transfer Embryos by Using G2/M-Stage Fetal Fibroblasts as Donors, BIOL REPROD 2001; 65: 1558-1564.

22. Liu J-L, et al. Refrigeration of Donor Cells in Preparation for Bovine Somatic Nuclear Transfer, REPROD 2001; 122: 801-808.

23. Meng L, et aL, Rhesus Monkeys Produced by Nuclear Transfer, BIOL REPROD 1997; 57: 454-459.

24. Park K W, et al., Developmental Potential of Porcine Nuclear Transfer Embryos Derived from Transgenic Fibroblasts Infected with the Gene for the Green Fluorescent Protein: Comparison of Different Fusion/Activation Conditions, BIOL REPROD 2001; 65: 1681-1685.

25. Polejaeva I A, et al., Cloned Pigs Produced by Nuclear Transfer from Adult Somatic Cells, NATURE 2000: 407: 86-90.

26. Ponimaskin E, et al., Sendai Virosomes Revisited: Reconstitution with Exogenous Lipids Leads to Potent Vehicles for Gene Transfer, VIROLOGY, 2000 April 10;269(2):391-403.

27. Reggio B C, et al,. Cloned Transgenic Offspring Resulting From Somatic Cell Nuclear Transfer in the Goat: Oocytes Derivedfrom Both Follicle-Stimulating Hormone-Stimulated and Nonstimulated Abattoir-Derived Ovaries, BIOL REPROD 2001; 65: 1528-1533.

28. Stice S L, et al., Pluripotent Bovine Embryonic Cell Lines Direct Embryonic Development Following Nuclear Transfer, BIOL REPROD. 1996 January; 54(1):100-10.

29. Stice S L and J M Robl, Nuclear Reprogramming in Nuclear Transplant Rabbit Embryo, BIOL REPROD 1998; 39(3): 657-64.

30. Verma P J, et al. In Vitro Development Of Porcine Nuclear Transfer Embryos Constructed Using Fetal Fibroblasts. MOL REPROD DEV 2000; 57: 262-269.

31. Wakayama T, et al., Full Term Development of Mice from Enucleated Oocytes Injected with Cumulus Cell Nuclei, NATURE 1998; 394: 369-374.

32. Wall R J, et al., Transgenic Dairy Cattle: Genetic Engineering on a Large Scale, J DAIRY SCI. 1997 September;80(9):2213-24.

33. Wells D N, et al., Production of Cloned Calves Following Nuclear Transfer with Cultured Adult Mural Granulosa Cells, Biol Reprod 1999; 60: 996-1005.

34. Willadsen S M, Nuclear Transplantation in Sheep Embryos, NATURE 1986; 320: 63-65.

35. Wilmut I, et al., Viable Offspring Derived From Fetal and Adult Mammalian Cells. NATURE 1997; 385: 810-813.

36. Yang X, S Jiang, A Kovacs and R H Foote, Nuclear Totipotency of Cultured Rabbit Morulae to Support Full-Term Development Following Nuclear Transfer, BIOL REPROD 1992; 47: 636-643.

37. Yong Z and L Yuqiang, Nuclear-Cytoplasmic Interaction and Development of Goat Embryos Reconstructed by Nuclear Transplantation: Production of Goats by Serially Cloning Embryos, BIOL REPROD 1998; 58: 266-269.

38. Zakhartchenko V, et al., Nuclear Transfer in Cattle Using in Vivo-Derived Vs. in Vitro-Produced Donor Embryos: Effect of Developmental Stage, MOL REPROD DEV 1996; 44: 493-498.

39. Zakhartchenko V, et al., Nuclear Transfer in Cattle with Non-Transfected and Transfected Fetal or Cloned Transgenic Fetal and Postnatal Fibroblasts, MOL REPROD DEV 2001; 60: 362-369.

40. Zou X, et al., Generation Of Cloned Goats (Capra Hircus) From Transfected Foetal Fibroblast Cells, The Effect Of Donor Cell Cycle, MOL REPROD DEV 2002; 61: 164-172.

What is claimed is:

1. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) activating a cell-couplet to create a transgenic embryo that is activated after an initial electrical shock;
(vii) culturing said activated first and/or second transgenic embryo(es) until greater than the 2-cell developmental stage; and
(viii) transferring said first and/or second transgenic embryo into a host mammal such that the embryo develops into a fetus;
(ix) wherein wherein the desired differentiated mammalian cell line to be used as a karyoplast is selected according to the objective parameters of cleavage and/or fusion patterns.

2. The method of claim 1, wherein said donor differentiated mammalian cell to be used as a source of donor nuclei or donor cell nucleus is from mesoderm.

3. The method of claim 1, wherein said donor differentiated mammalian cell to be used as a source of donor nuclei or donor cell nucleus is from endoderm.

4. The method of claim 1, wherein said donor differentiated mammalian cell to be used as a source of donor nuclei or donor cell nucleus is from ectoderm.

5. The method of claim 1, wherein said donor differentiated mammalian cell to be used as a source of donor nuclei or donor cell nucleus is from fetal somatic tissue.

6. The method of claim 1, wherein said donor differentiated mammalian cell to be used as a source of donor nuclei or donor cell nucleus is from fetal somatic cells.

7. The method of claim 1, wherein said donor differentiated mammalian cell to be used as a source of donor nuclei or donor cell nucleus is from a fibroblast.

8. The method of claim 1, wherein said donor differentiated mammalian cell to be used as a source of donor nuclei or donor cell nucleus is from an ungulate.

9. The method of either claims 1 or 8, wherein said donor cell or donor cell nucleus is from an ungulate selected from the group consisting of bovine, ovine, porcine, equine, caprine and buffalo.

10. The method of claim 1, wherein said donor differentiated mammalian cell to be used as a source of donor nuclei or donor cell nucleus is from an adult non-human mammalian somatic cell.

11. The method of claim 1, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

12. The method of claim 1, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

13. The method of claim 1, wherein said at least one oocyte is matured in vivo prior to enucleation.

14. The method of claim 1, wherein said at least one oocyte is matured in vitro prior to enucleation.

15. The method of claim 1, wherein said non-human mammal is a rodent.

16. The method of claim 1, wherein said donor differentiated mammalian cell to be used as a source of donor nuclei or donor cell nucleus is a non-quiescent somatic cell or a nucleus isolated from said non-quiescent somatic cell.

17. The method of either claims 1 or 8, wherein the fetus develops into an offspring.

18. The method of claim 1, wherein said at least one oocyte is enucleated about 10 to 60 hours after initiation of in vitro maturation.

19. The method of claim 1, wherein a desired gene is inserted, removed or modified in said differentiated mammalian cell or cell nucleus prior to insertion of said differentiated mammalian cell or cell nucleus into said enucleated oocyte.

20. The resultant offspring of the methods of claims 1 or 19.

21. The resultant offspring of claim 19 further comprising wherein the offspring created as a result of said nuclear transfer procedure is chimeric.

22. The method of claim 1, wherein cytocholasin-B is used in the cloning protocol.

23. The method of claim 1, wherein cytocholasin-B is not used in the cloning protocol.

24. A method for producing cultured inner cell mass cells, comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) activating a cell-couplet to create a first transgenic embryo that is activated after an initial electrical shock; and
(vi) culturing cells obtained from said cultured activated embryo to obtain cultured inner cell mass cells;
(vii) wherein the desired differentiated mammalian cell line to be used as a karyoplast is selected according to the objective parameters of cleavage and/or fusion patterns

25. The method of claim 24, wherein said donor differentiated mammalian cell to be used as a source of donor nuclei or donor cell nucleus is from mesoderm.

26. The method of claim 24, wherein said donor differentiated mammalian cell to be used as a source of donor nuclei or donor cell nucleus is from endoderm.

27. The method of claim 24, wherein said donor differentiated mammalian cell to be used as a source of donor nuclei or donor cell nucleus is from ectoderm.

28. The method of claim 24, wherein said donor differentiated mammalian cell to be used as a source of donor nuclei or donor cell nucleus is from fetal somatic tissue.

29. The method of claim 24, wherein said donor differentiated mammalian cell to be used as a source of donor nuclei or donor cell nucleus is from fetal somatic cells.

30. The method of claim 24, wherein said donor differentiated mammalian cell to be used as a source of donor nuclei or donor cell nucleus is from a fibroblast.

31. The method of claim 24, wherein said donor differentiated mammalian cell to be used as a source of donor nuclei or donor cell nucleus is from an ungulate.

32. The method of either claims 24 or 31, wherein said donor cell or donor cell nucleus is from an ungulate selected from the group consisting of bovine, ovine, porcine, equine, caprine and buffalo.

33. The method of claim 24, wherein said donor differentiated mammalian cell to be used as a source of donor nuclei or donor cell nucleus is from an adult mammalian somatic cell.

34. The method of claim 24, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

35. The method of claim 24, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

36. The method of claim 24, wherein said at least one oocyte is matured in vivo prior to enucleation.

37. The method of claim 24, wherein said at least one oocyte is matured in vitro prior to enucleation.

38. The method of claim 24, wherein said mammalian cell is derived from a rodent.

39. The method of claim 24, wherein said donor differentiated mammalian cell to be used as a source of donor nuclei or donor cell nucleus is a non-quiescent somatic cell or a nucleus isolated from said non-quiescent somatic cell.

40. The method of either claims 24 or 31, wherein any of said cultured inner cell mass cells fetus develops into a non-human offspring.

41. The method of claim 24, wherein said at least one oocyte is enucleated about 10 to 60 hours after initiation of in vitro maturation.

42. The method of claim 24, wherein a desired gene is inserted, removed or modified in said differentiated mammalian cell or cell nucleus prior to insertion of said differentiated mammalian cell or cell nucleus into said enucleated oocyte.

43. The resultant offspring of the methods of claims 24 or 42.

44. The resultant offspring of claim 42 further comprising wherein any non-human offspring created as a result of said nuclear transfer procedure is chimeric.

45. The method of claim 24, wherein cytocholasin-B is used in the protocol.

46. The method of claim 24, wherein cytocholasin-B is not used in the protocol.

47. The method of claim 24, wherein cytocholasin-B is used in the protocol.

48. The method of claim 24, wherein said cultured inner cell mass cells are used to develop a functional organ for transplantation.

49. The method of claim 24, wherein said cultured inner cell mass cells are used in organogenesis.

50. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said oocytes;
(iv) transferring the desired differentiated cell or cell nucleus into the enucleated oocyte;
employing at least two electrical shocks to a cell-couplet to initiate fusion and activation of said cell-couplet into an activated and fused embryo.
(vii) culturing said activated and fused embryo until greater than the 2-cell developmental stage;
(viii) transferring said first and/or second transgenic embryo into a host mammal such that the embryo develops into a fetus;
wherein the second of said at least two electrical shocks is administered at least 15 minutes after an initial electrical shock;
wherein a desired gene is inserted, removed or modified in said differentiated mammalian cell or cell nucleus prior to insertion of said differentiated mammalian cell or cell nucleus into said enucleated oocyte; and
wherein the desired differentiated mammalian cell line to be used as a karyoplast is selected according to the objective parameters of cleavage and/or fusion patterns.

51. An improved method of cloning a non-human mammal by nuclear transfer comprising the introduction of a non-human mammalian donor cell or a non-human mammalian donor cell nucleus into a non-human mammalian enucleated oocyte of the same species as the donor cell or donor cell nucleus to form a nuclear transfer (NT) unit, implantation of the NT unit into the uterus of a surrogate mother of said species, and permitting the NT unit to develop into the cloned mammal, wherein the improvement comprises utilizing a pre-screened differentiated mammalian cell line as a karyoplast, said karyoplast being selected according to successful cleavage patterns.

52. An improved method of cloning a non-human mammal by nuclear transfer comprising the introduction of a non-human mammalian donor cell or a non-human mammalian donor cell nucleus into a non-human mammalian enucleated oocyte of the same species as the donor cell or donor cell nucleus to form a nuclear transfer (NT) unit, implantation of the NT unit into the uterus of a surrogate mother of said species, and permitting the NT unit to develop into the cloned mammal, wherein the improvement comprises utilizing a pre-screened differentiated mammalian cell line as a karyoplast, said karyoplast being selected according to successful fusion patterns.

53. An improved method of cloning a non-human mammal by nuclear transfer comprising the introduction of a non-human mammalian donor cell or a non-human mammalian donor cell nucleus into a non-human mammalian enucleated oocyte of the same species as the donor cell or donor cell nucleus to form a nuclear transfer (NT) unit, implantation of the NT unit into the uterus of a surrogate mother of said species, and permitting the NT unit to develop into the cloned mammal, wherein the improvement comprises utilizing a pre-screened differentiated mammalian cell line as a karyoplast, said karyoplast being selected according to successful cleavage and fusion patterns.
